(19) United States
(12) Patent Application Publication
(10) Pub. No.: US 2023/0055867 A1
(43) Pub. Date: Feb. 23, 2023

(54) METHOD AND APPARATUS FOR PERFORMING SPATIAL FILTERING AND AUGMENTING ELECTROENCEPHALOGRAM SIGNAL, ELECTRONIC DEVICE, AND STORAGE MEDIUM

(71) Applicant: TIANJIN UNIVERSITY, Tianjin (CN)

(72) Inventors: Minpeng XU, Tianjin (CN); Qiaoyi WU, Tianjin (CN); Ruixin LUO, Tianjin (CN); Dong MING, Tianjin (CN)

(21) Appl. No.: 17/904,790

(22) PCT Filed: Jul. 9, 2021
(86) PCT No.: PCT/CN2021/105590

§ 371 Date: Aug. 23, 2022

(30) Foreign Application Priority Data
Jul. 24, 2020 (CN) 202010728201.9
Apr. 20, 2021 (CN) 202110426679.0

(51) Int. Cl. A61B 5/00; A61B 5/02

(52) U.S. Cl. CPC A61B 5/0002; A61B 5/02; G06F 3/015

(57) ABSTRACT

A method for performing spatial filtering and augmenting an electroencephalogram (EEG) signal is provided. A processor constructs a spatial filter based on channel information of the EEG signal. The processor augments the EEG signal with the spatial filter. A related electronic device and a related non-transitory computer-readable storage medium are provided.

### CROSS REFERENCE TO RELATED APPLICATIONS

This disclosure is a U.S. national phase application of International Patent Application No. PCT/CN2021/105590, filed on Jul. 9, 2021, which claims priority to Chinese Patent Application No. 202010728201.9, filed on Jul. 24, 2020, and Chinese Patent Application No. 202110426679.0, filed on Apr. 20, 2021. The entire disclosures of the above-identified applications are incorporated herein by reference.

### TECHNICAL FIELD

The disclosure relates to a field of computer technologies, a field of brain-computer interface technologies, a field of brain function cognitive state evaluation technologies, and a field of brain state detection technologies, particularly to a method and a device for performing dynamic spatial filtering and augmenting an Electroencephalogram signal, an electronic device, and a storage medium.

### BACKGROUND

Electroencephalogram (EEG) signal is an overall reflection of electrophysiological activities of brain nerve cells on the cerebral cortex, which can be recorded by scalp electrodes. How to augment the EEG signal is a problem to be solved.

### SUMMARY

According to the first aspect, a method for performing dynamic spatial filtering and augmenting an EEG signal is provided. The method includes constructing a spatial filter based on channel information of the EEG signal; and augmenting the EEG signal with the spatial filter.

According to a second aspect, there is provided an electronic device, including: at least one processor; and a memory, communicating with the at least one processor, in which the memory is configured to store instructions executable by the at least one processor, and the instructions are configured to cause the at least one processor to execute a method as described above when being executed by the at least one processor.

According to a third aspect, there is provided a non-transitory computer-readable storage medium having computer instructions stored thereon, in which the computer instructions are configured to cause a computer to execute a method as described above.

### BRIEF DESCRIPTION OF THE DRAWINGS

FIG. 1A is a schematic diagram illustrating an arrangement of electrodes for collecting EEG signals in the related art.

FIG. 1B is a schematic diagram of processing an EEG signal in the related art.

FIG. 1C is a schematic diagram of processing an EEG signal according to some embodiments of the disclosure.

FIG. 2 is a flowchart according to an embodiment of the disclosure.

FIG. 3 is a flowchart according to another embodiment of the disclosure.

FIG. 4 is a flowchart according to still another embodiment of the disclosure.

FIG. 5 is a schematic diagram illustrating division of an EEG signal through a dynamic time window according to an embodiment of the disclosure.

FIG. 6 is an overall flowchart according to an embodiment of the disclosure.

FIG. 7 is a flowchart according to another embodiment of the disclosure.

FIG. 8 is a flowchart according to still another embodiment of the disclosure.

FIG. 9 is a block diagram illustrating an electronic device for executing an EEG signal augmentation method according to embodiments of the disclosure.

### DETAILED DESCRIPTION

Embodiments of the disclosure will be described in detail below, and examples of these embodiments are shown in accompanying drawings, in which the same or similar reference numerals throughout the disclosure represent the same or similar elements, or elements with the same or similar functions. The embodiments described below with reference to the accompanying drawings are exemplary, are only used to explain the disclosure, and cannot be understood as limitations to the disclosure. On the contrary, the embodiments of the disclosure include all changes, modifications and equivalents that fall within the spirit and scope of the appended claims.

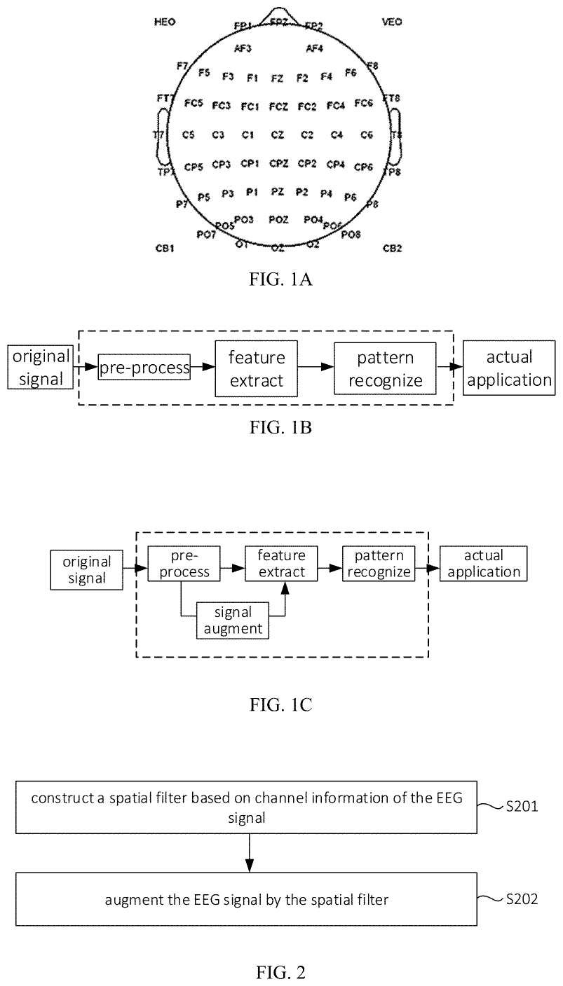

Electroencephalogram (EEG) signal is an overall reflection of electrophysiological activities of brain nerve cells on the cerebral cortex, which can be recorded by scalp electrodes (FIG. 1A illustrates a schematic diagram of the arrangement of electrodes). The EEG signal contains a large amount of physiological information. In engineering applications, the EEG signal can be used to implement a brain-computer interface (BCI). The BCI is a new human-computer interaction system that can obtain and decode the physiological signals generated by the human brain to control computers or external devices, which can be independent from a normal command output pathway of the brain and does not need to use a traditional motion control pathway via peripheral nerves and related muscle tissues. Depending on the stimulus manner, the BCI system can be classified into an active BCI, a passive BCI, or a reactive BCI. Characteristics of the active BCI include that a user actively outputs a command to control external devices, which is mainly a system based on a motor imagery (MI) signal. The passive BCI is mainly used to detect states of the brain, such as mental state and attention level. The reactive BCI is mainly used to detect responses of the brain to an external stimulus and indirectly output a control command. There are many kinds of stimulus-evoked signals, such as event-related potential (ERP), steady state visual evoked potential (SSVEP), error-related potential (ErrP), event-related desynchronization (ERD), and the like. The BCI system is suitable for the following two application scenarios. One is patients with impaired basic limb motor function but normal thinking; and the other one is narrow working space that is inconvenient for movement of limbs (such as an aerospace environment). At present, the BCI technology is paid more and more attention.

The EEG signal is a non-stationary and time-varying random signal, and is easily disturbed by background activity noise, motion artifact, electromagnetic noise and so on. In order to reduce noise interference and improve the signal-to-noise ratio of effective signals, most collected EEG signals need to undergo various preprocessing before a next-step processing. For example, downsampling can reduce storage pressure, improve real-time operation speed, as well as suppress the interference of high-frequency noise to a certain extent. Digital filtering is used to filter or retain signals in specific frequency bands. Types of the digital filtering can include low-pass filtering, high-pass filtering, band-pass filtering and notch filtering. Signal-space projection (SSP) is used to eliminate electromagnetic noise generated by equipment and eye electrical interference. Independent component analysis (ICA) is used to separate non-Gaussian statistically independent source signals. Principal component analysis (PCA) is used to perform data dimensionality reduction and extract main features of signals. Maxwell filtering and signal-space separation (SSS) are used to separate and remove electromagnetic noise (environmental noise) from external sources.

In recent years, the EEG signal and the applications of the EEG signals have been widely and deeply studied. The number of instruction sets of the BCI system is increasing, and the information transfer rate (ITR) is gradually improving. However, at present, relevant researches and developments have reached their bottlenecks in both directions of improving response time and increasing accuracy. One reason is that the above-mentioned EEG data preprocessing methods are not enough to further improve the quality of characteristic signals, and specific parameters of existing spatial filters are fixed in advance based on training set data or corresponding prior knowledge. Therefore, the non-target features, EEG noises, with strong randomness, nonlinearity and non-stationarity cannot be well processed.

In addition, existing signal processing methods for the EEG signals include preprocessing, feature extraction and pattern recognition (illustrated in the dotted box in FIG. 1B). The signal preprocessing can suppress the noise signal, which is helpful for feature extraction, classification and recognition. However, the signal preprocessing cannot increase effective component information of the signal. In recent years, the BCI system, which obtains information of the brain through the EEG signal, has developed rapidly, and its performance has been continuously optimized. However, there are still some limitations in decoding the EEG signals, such as low spatial resolution and small data amount. In order to make effective use of existing EEG data, researchers expect to increase the effective information of the signal through data augmentation. The EEG signal is a multi-channel dynamic time series, and traditional geometric transformation methods of image enhancement are not suitable to apply to the EEG signal. At present, the EEG signal augmentation methods in the research have poor effect and robustness.

In view of the above-mentioned technical problem that the method for augmenting an electroencephalogram (EEG) signal in the related art has poor effect and robustness such that the method cannot be widely used in actual construction of a brain-computer interface (BCI) system, embodiments of the disclosure provide a method for augmenting an EEG signal.

With the technical solution according to embodiments of the disclosure, the EEG signal is filtered by constructing and applying the spatial filters to obtain the augmented signals, and the augmented signals are spliced and integrated to realize the augmentation of the EEG signal. Therefore, potential EEG information can be effectively discovered from the original EEG signal, reflecting the current EEG characteristics, realizing the augmentation of the EEG signal, and improving the reliability and effectiveness of the EEG information. Further, the technical problem that the existing method for augmenting an EEG signal has poor effect and robustness such that it has not been widely used in the actual BCI system can be solved.

In addition, beneficial effects of the technical solution according to embodiments of the disclosure further include the following.

1. The spatial filler can be dynamically designed in preprocessing the EEG signal to suppress a variety of non-target features, i.e., the EEG noise, thereby having a wide range of applications.

2. Analysis of experimental data of the brain-computer interface (BCI) shows that the signal-to-noise ratio of the single-trial EEG characteristic signal can be significantly improved, the recognition accuracy of subsequent feature classification can be effectively improved, the preprocessing technology of the EEG signal can be further improved, and the transformation of the technology to the application achievement can be promoted;

3. The data preprocessing of the EEG signal can effectively improve the quality of collected signals, improve the performance of the BCI system, and considerable social and economic benefits can be achieved.

As illustrated in FIG. 1C, before extracting features of the EEG signal, the method according to embodiments of the disclosure can augment the EEG signal, which will be described in detail below.

It is to be noted that an execution subject of the method for augmenting an EEG signal according to embodiments of the disclosure may be a device for augmenting an EEG signal. The device may be implemented by software and/or hardware. The device may be included in an electronic device. The electronic device may include, but is not limited to, a terminal or a server.

FIG. 2 is a flowchart according to an embodiment of the disclosure. As illustrated in FIG. 2, the method for augmenting an EEG signal includes the following.

At block S201, a spatial filter is generated based on channel information of the EEG signal.

In this embodiment, the channel information of the EEG signal is obtained. The EEG signal may include multiple channels, such as three channels, i.e., FP1, C3, and O1. Information of the multiple channels may be referred to as the channel information of the EEG signal.

After obtaining the channel information, the spatial filter can be constructed based on the channel information. The construction of the spatial filter can be understood as dynamically solving for a spatial filter suitable for a current environment. The spatial filter can be constructed in any possible way, which is not limited in the disclosure.

At block S202, the EEG signal is augmented by the spatial filter.

After the spatial filter is constructed, the spatial filter can be used to augment the EEG signal, effectively discovering potential EEG information from the original EEG signal to reflect current EEG characteristics, thereby realizing the augmentation of the EEG signal. Therefore, the reliability and validity of the EEG information are improved.

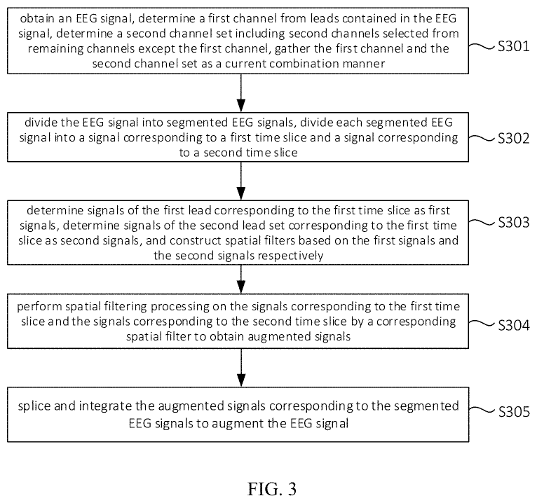

FIG. 3 is a flowchart according to another embodiment of the disclosure. As illustrated in FIG. 3, the method for augmenting an EEG signal includes the following.

At block S301, the EEG signal is obtained, a first channel is determined from multiple channels included in the EEG signal, a second channel set including at least one second channel selected from remaining channels of the multiple channels except the first channel is determined, and the first channel and the second channel set are gathered as a current combination manner.

In detail, the EEG signal can be obtained. The EEG signal is, for example, but not limited to, an epilepsy EEG signal or a steady-state visual evoked potential signal. The EEG signal may be an originally collected EEG signal or a preprocessed EEG signal. The EEG signal can be a two-dimensional signal X∈R^{N}^{_{c}}^{×N}^{ₜ}, where N_{c} denotes the number of channels of the EEG signal and is a real number, Nₜ denotes the number of sampling time points and is a real number, and R denotes a real number set.

Further, the first channel is determined from the multiple channels included in the EEG signal, and a second channel set is formed by selecting at least one second channel from the remaining channels except the first channel. In an example, taking the epilepsy EEG signal as an example, the epilepsy EEG signal includes, for example three channels, i.e., FP1, C3, and O1. The first channel and the second channel set can be determined from these three channels. In actual operations, the first channel (e.g., the FP1) can be determined and the second channel set including the arbitrary number of second channels extracted from the remaining channels except the first channel is determined. Thus, in this example, the second channel set is one of {C3,O1}, {C3}, and {O1}.

In addition, the current combination manner corresponding to the EEG signal also needs to be determined. The current combination manner refers to a combination manner between the first channel and the second channel set. In the above example, all combination manners for the case where the first channel and the second channel set are selected from FP1, C3 and O1 are shown in Table 1:

TABLE 1

| | | second channel set | | | | | |
|---|---|---|---|---|---|---|---|
| | | {C3, | {C3, | {O1, | | | |
| | | O1} | FP1} | FP1} | {FP1} | {C3} | {O1} |
| first | FP1 | ✓ | | | | ✓ | ✓ |
| channel | C3 | | | ✓ | ✓ | | ✓ |
| | O1 | | ✓ | | ✓ | ✓ | |

As shown in Table 1, nine combination manners, i.e., FP1 and {C3,O1}, FP1 and {C3}, FP1 and {O1}, C3 and {FP1,O1}, C3 and {FP1}, C3 and {O1}, O1 and {FP1, C3}, O1 and {FP1}, O1 and {C3} are included. One of the nine combination manners can be determined as the current combination manner. As an example, the current combination manner is FP1 and {C3, O1}. Embodiments of the disclosure will be described in detail below by taking FP1 and {C3, O1} as the current combination manner.

It is to be understood that although the EEG signal is taken as an example to describe embodiments of the disclosure, those skilled in the art can also apply the method to other application scenarios, such as an electrocardiograph (ECG) signal. The format of the signal is not limited herein.

At block S302, the EEG signal is divided into multiple segmented EEG signals, and each of the segmented EEG signals is divided into a signal corresponding to a first time slice and a signal corresponding to a second time slice.

In detail, after the EEG signal is acquired, the EEG signal needs to be divided into multiple segmented EEG signals. That is, a piece of EEG signal is cut into multiple segmented EEG signals. For example, the EEG signal is divided into a segmented EEG signal 1, a segmented EEG signal 2, . . . a segmented EEG signal j, where j represents a serial number of the segmented EEG signal.

Further, the multiple segmented EEG signals are each divided into a respective signal corresponding to the first time slice or a respective signal corresponding to the second time slice. In actual operations, any one segmented EEG signal can be arbitrarily selected from the multiple segmented EEG signals, and this selected segmented EEG signal is divided again based on the time to obtain the signal corresponding to the first time slice and the signal corresponding to the second time slice. The length of the first time slice and the length of the second time slice may be determined according to actual needs. The lengths can be the same or different, which is not limited in the disclosure. Each segmented EEG signal can be divided in the above-mentioned manner, which is not repeated here. Therefore, for each segmented EEG signal, the respective signal corresponding to the first time slice and the respective signal corresponding to the second time slice can be obtained.

At block S303, signals of the first channel corresponding to the first time slice are determined as first signals, signals of the second channel set corresponding to the first time slice are determined as second signals, and spatial filters are constructed based on the first signals and the second signals respectively.

In detail, after the signals corresponding to the first time slice and the signals corresponding to the second time slice are determined, the signals of the first channel corresponding to the first time slice are determined as the first signals, and the signals of the second channel set corresponding to the first time slice are determined as the second signals.

In actual operations, for each segmented EEG signal, the signal of the first channel corresponding to the first time slice is determined as the first signal, and the signal of the second channel set corresponding to the first time slice is determined as the second signal.

In an example, for the segmented EEG signal j, the signal of the first channel FP1 corresponding to the first time slice of the segmented EEG signal j is determined as the first signal, and the signal of the second channels C3 and O1 corresponding to the first time slice of the segmented EEG signal j is determined as the second signal. The determination of the first signal and the second signal for other segmented EEG signals can be performed in the same way of determining the first signal and the second signals for the segmented EEG signal j, which is not repeated herein. Therefore, for each segmented EEG signal (i.e., the segmented EEG signal 1, . . . the segmented EEG signal j), the first signal of the first channel of the segmented EEG signal and the second signal of the second channel of the segmented EEG signal can be determined.

Further, corresponding spatial filters can be constructed respectively based on the multiple first signals and the multiple second signals. That is, a spatial filter is separately constructed based on the first signal and the second signal(s) determined for each segmented EEG signal, such that each segmented EEG signal corresponds to a respective spatial filter. Principles of constructing the spatial filter can be the same as the principles of constructing the spatial filter in the related art. The spatial filter is not limited herein.

At block S304, the signals corresponding to the first time slice and the signals corresponding to the second time slice are spatially filtered respectively by the multiple spatial filters, to obtain augmented signals.

Further, when the construction of the spatial filters finishes, the multiple spatial filters are used to perform spatial filtering processing on the respective signals corresponding to the first time slice and the respective signals corresponding to the second time slice. That is, the spatial filter is used to filter the signals corresponding to the first time slice and the signals corresponding to the second time slice of a corresponding segmented EEG signal, to obtain augmented signals corresponding to the first time slice and augmented signals corresponding to the second time slice of the corresponding segmented EEG signal.

At block S305, multiple augmented signals corresponding to the multiple segmented EEG signals are spliced and integrated to augment the EEG signal.

Finally, when the augmented signals are obtained, the multiple augmented signals corresponding to the multiple segmented EEG signals are spliced and integrated. In an implementation, the augmented signals corresponding to different time slices of a segmented EEG signal can be spliced. After the augmented signal corresponding to the first time slice and the augmented signal corresponding to the second time slice are spliced for each segmented EEG signal, spliced augmented signals of the multiple segmented EEG signals are integrated to finish the augmentation process of the EEG signal.

With the technical solution according to embodiments of the disclosure, the EEG signal is filtered by constructing and applying the spatial filters to obtain the augmented signals, and the augmented signals are spliced and integrated to realize the augmentation of the EEG signal. Therefore, potential EEG information can be effectively discovered from the original EEG signal, reflecting the current EEG characteristics, realizing the augmentation of the EEG signal, and improving the reliability and effectiveness of the EEG information. Further, the technical problem that the existing method for augmenting an EEG signal has poor effect and robustness such that it has not been widely used in the actual BCI system can be solved.

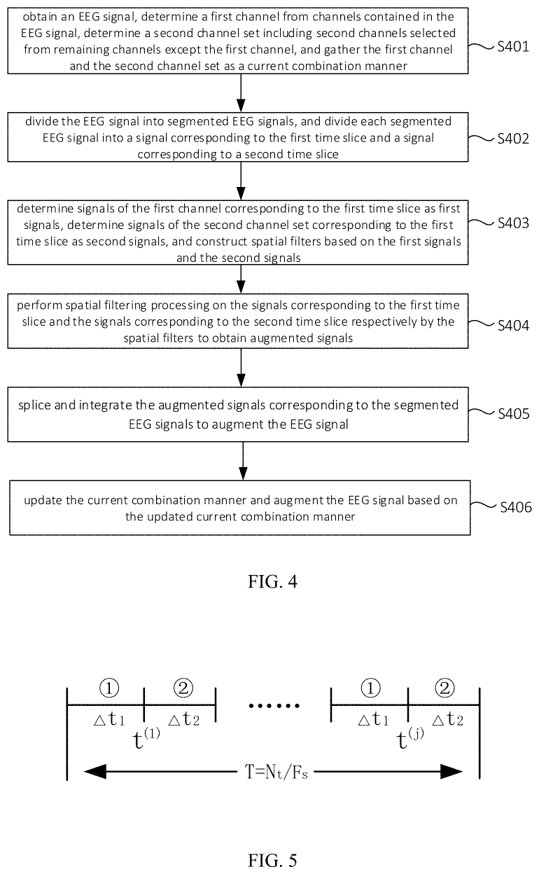

In the above embodiment, the augmentation of the EEG signal based on one combination manner of channels (e.g., FP1 and {C3, O1}) is realized. However, in order to further augment the EEG signal, a second embodiment is also provided in the disclosure. FIG. 4 is a schematic diagram according to another embodiment of the disclosure. As illustrated in FIG. 4, the method for augmenting an EEG signal includes the following.

At block S401, an EEG signal is obtained, a first channel is obtained from multiple channels included in the EEG signal, and a second channel set including at least one second channel selected from the multiple channels except the first channel is determined, and the first channel and the second channel set are gathered as a current combination manner.

At block S402, the EEG signal is divided into a plurality of segmented EEG signals, and each of the plurality of segmented EEG signals is divided into a signal corresponding to a first time slice and a signal corresponding to the second time slice.

At block S403, signals of the first channel corresponding to the first time slice are determined as first signals, signals of the second channel set corresponding to the first time slice are determined as second signal, and a plurality of spatial filters are constructed based on the first signals and the second signals respectively.

At block S404, the plurality of spatial filters are used to perform spatial filtering processing on respective signals corresponding the first time slice and respective signals corresponding to the second time slice respectively, to obtain augmented signals.

At block S405, multiple augmented signals corresponding to the multiple segmented EEG signals are spliced and integrated to augment the EEG signal.

Descriptions of the blocks S401-S405 can make reference to the above-mentioned embodiment, and details are not repeated here.

At block S406, the current combination manner is updated and the EEG signal corresponding to the updated current combination manner is augmented.

In detail, in combination with the above-mentioned embodiment, after the signal augmentation is completed for the current combination manner (i.e., FP1 and {C3, O1}), the current combination manner can be updated. In actual operations, the above nine combination manners are traversed, and each combination manner can be used as the current combination manner to perform the blocks S402 to S405.

In an example, after the signal augmentation is completed for the current combination manner (e.g., FP1 and {C3, O1}), the remaining 8 combination manners are traversed. When the traversal proceeds to the combination manner, e.g., FP1 and {C3}, the combination manner FP1 and {C3} is used as the updated current combination manner. Further, the blocks S402-S405 are performed for the updated current combination manner FP1 and {C3}, and the augmentation of the EEG signal of the combination manner FP1 and {C3} is completed. The combination manners are traversed in turn, until the EEG signals of the nine combination manners are all augmented.

Given the augmented signal obtained based on a combination manner is represented by y⁽ⁿ⁾, where n denotes a serial number of the combination manner, the augmented signals obtained based on the above-mentioned nine combination manners are shown in Table 2:

TABLE 2

| | | second channel set | | | | | |
|---|---|---|---|---|---|---|---|
| | | {C3, | {C3, | {O1, | | | |
| | | O1} | FP1} | FP1} | {FP1} | {C3} | {O1} |
| first | FP1 | y⁽¹⁾ | | | | y⁽²⁾ | y⁽³⁾ |
| channel | C3 | | | y⁽⁴⁾ | y⁽⁵⁾ | | y⁽⁶⁾ |
| | O1 | | y⁽⁷⁾ | | y⁽⁸⁾ | y⁽⁹⁾ | |

In this way, more augmented signals can be obtained, to realize the augmentation of the EEG signal.

In some examples, dividing the EEG signal into a plurality of segmented EEG signals and dividing each of the segmented EEG signals into a respective signal corresponding to the first time slice and a respective signal corresponding to a second time slice consecutively after the first time slice, including: dividing the EEG signal into the plurality of segmented EEG signals by a dynamic time window, where the dynamic time window is denoted by a time range [t−Δt₁,t+Δt₂] centered on t, [t−Δt₁,t] denotes the first time slice and [t,t+Δt₂] denotes the second time slice.

In detail, in the third embodiment of the disclosure, in dividing the EEG signal into the plurality of segmented EEG signals, and dividing each segmented EEG signal into the respective signal corresponding to the first time slice and the respective signal corresponding to the second time slice continuously after the first time slice, the dynamic time window can be used.

In an example, as illustrated in FIG. 5, the dynamic time window is [t−Δt₁,t+Δt₂], representing a dynamic time window centered on t. Elements t^{(j)} in a set of center points t={t⁽¹⁾, t⁽²⁾ . . . t^{(j)})} of the time window represent center points of respective segmented EEG signals obtained by the division, which satisfy a condition of Δt₁≤t^{(j)}≤T−Δt₂, a step size between different center points of the time window is denoted as tₛ; j denotes a serial number of segmented EEG signals, i.e., t⁽¹⁾, t⁽²⁾ . . . t^{(j)} respectively correspond to the above-mentioned segment EEG signal 1, segment EEG signal 2, . . . segmented EEG signal j; and T denotes a total duration of the EEG signal.

In addition, the segmented EEG signals can be divided by this dynamic time window. In detail, as illustrated in FIG. 5, [t−Δt₁,t] denotes the first time slice (i.e., the time slice \textcircled{1} in FIG. 5), and [t,t+Δt₂] denotes the second time slice (i.e., the time slice \textcircled{2} in FIG. 5).

In this way, the EEG signal can be accurately divided to obtain the segmented EEG signals, such that the process of dividing each segmented EEG signal into the signal corresponding to the first time slice and the signal corresponding to second time slice is easy.

In some embodiments, in constructing the spatial filters, a following target equation can be used:

$${\hat{W}}_{j} = {argmin}_{{W}_{j}} \{ {‖ {W}_{j} * {U}_{j} ( {ς}_{i} , : ) - {U}_{j} ( i , : ) ‖}_{p} \}$$

this target equation is a constraint condition of a spatial filter Wⱼ corresponding to the segmented EEG signal j, where ∥*∥ₚ is the p-norm of a vector, the argmin function is to search for a variable value that minimizes a target, Ŵⱼ is an estimation of the spatial filter Wⱼ under the constraint condition, Uⱼ(i,:) is the first signal, i denotes the serial number of the first channel; Uⱼ(çᵢ,:) is the second signal, çᵢ denotes the second channel set, Uⱼ∈R^{N}^{_{c}}^{×m} denotes the signal corresponding to the first time slice of the segmented EEG signal having the serial number of j, N_{c} denotes the number of channels included in the EEG signal, m denotes the number of sampling points within the dynamic time window, m=[Δt₁×Fₛ], m is an integer not exceeding the real number, and Fₛ is a sampling frequency of the EEG signal.

By constructing the spatial filters based on the EEG signal, the spatial filters can fully retain the characteristics of the EEG signal, thereby improving the robustness of the augmentation process of the EEG signal such that the augmented EEG signal can well reflect the current EEG characteristics.

It is to be understood that the above target equation is used as an example to explain the process of constructing the spatial filters, the construction of the spatial filters is not limited to using the above target equation, and other equations or other methods can be used, which is not limited here.

In some embodiments, in performing the spatial filtering processing on the signal corresponding to the first time slice and the signal corresponding to the second time slice of each segmented EEG signal by the spatial filter to obtain the augmented signals, following equations can be used:

χⱼ=Ŵⱼ*Uⱼ(çᵢ,:)−Uⱼ(i,:), and

γⱼ=Ŵⱼ*Vⱼ(çᵢ,:)−Vⱼ(i,:),

where, χⱼ∈R^{1×m} and γⱼ∈R^{1×n} denotes the augmented signals obtained by filtering Uⱼ and Vⱼ respectively, Vⱼ∈R^{N}^{_{c}}^{×n} denotes the signal corresponding to the second time slice of the segmented EEG signal having the serial number of j, n represents the number of sampling points within the dynamic time window, n=[Δt₂×Fₛ], n is an integer not exceeding the real number, Vⱼ(i,:) denotes the signal of the first channel having the serial number of i corresponding to the second time slice of the segmented EEG signal having the serial number of j, and Vⱼ(çᵢ,:) denotes the signal of the second channel set corresponding to the second time slice of the segmented EEG signal having the serial number of j, where the second channel set corresponds to the first channel having the serial number of i.

In this way, the signal corresponding to each time slice can be augmented separately, such that the obtained augmented signal can reflect the current EEG characteristics.

It is to be understood that the above equations are only used as an example to explain the signal augmentation process, but the signal augmentation process is not limited to the above-mentioned method and other equations or other method can also be used to augment the signal, which is not limited here.

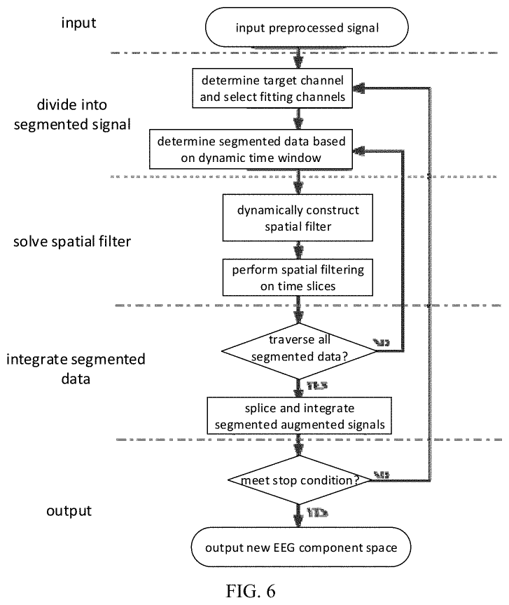

In order to describe the technical solution of the disclosure clearly, the technical solution will be further explained with an embodiment providing an overall process. FIG. 6 is a schematic diagram illustrating an overall process according to embodiments of the disclosure. As illustrated FIG. 6, the overall process includes the following.

1) A pre-processed signal (e.g., the EEG signal in the above embodiments) is input.

2) A target channel (e.g., the first channel in the above embodiments) is determined, and a channel set (e.g., the second channel set in the above embodiments) including the arbitrary number of channels selected from the remaining channels except the target channel is determined. For the pre-processed data, several pieces of segmented data (e.g., the segmented EEG signals in the above embodiments) are sequentially obtained through cutting by a dynamic time window, and each piece of segmented data is divided into signals corresponding to two time slices (e.g., the first time slice and the second time slice in the above embodiments) respectively.

The input preprocessed data can be represented as a two-dimensional signal X∈R^{N}^{_{c}}^{×N}^{ₜ}, where N_{c} and Nₜ denote the number of channels of the EEG signal and the number of data points (e.g., the sampling time points) respectively, which are both constants, R denotes a real number set. As illustrated in FIG. 5, the time range [t−Δt₁,t+Δt₂] denotes the dynamic time window with the center point t. Elements t^{(j)} in the set of center points t={t⁽¹⁾, t⁽²⁾ . . . t^{(j)}} of the time window need to meet a condition Δt₁≤t^{(j)}≤T−Δt₂, a step size of different center points of the time window is denoted as tₛ, j denotes a serial number of the segmented data, and T denotes a total duration of the signal.

The dynamic time window can divide the segmented data into a signal corresponding to the time slice \textcircled{1} (e.g., the first time slice in the above embodiments) within [t−Δt₁,t], which can be denoted as Uⱼ∈R^{N}^{_{c}}^{×m} for the segmented data having the serial number of j, and a signal corresponding to the time slice \textcircled{2} (e.g., the second time slice in the above embodiments) within [t,t+Δt₂], which can be denoted as Vⱼ∈R^{N}^{_{c}}^{×n} for the segmented data having the serial number of j, where m and n denote the number of sampling points within the dynamic time window, which can be determined through the following equations (1) and (2):

m=[Δt₁×Fₛ], where m is a positive integer, and (1)

n=[Δt₂×Fₛ], where n is a positive integer, (2)

where, Fₛ is a sampling frequency of the signal; [x] is a rounding function, which means an integer not exceeding the real number x.

3) For any one piece of segmented data, a template signal (e.g., the first signal in the above embodiments) of the target channel and a fitting signal (e.g., the second signal in the above embodiments) of the selection channel set corresponding to the two time slices respectively are determined. A dynamic spatial filter is constructed based on the template signal and the fitting signal corresponding to a previous time slice.

The construction of the spatial filter can be denoted as follows:

$${\hat{W}}_{j} = {argmin}_{{W}_{j}} \{ {‖ {W}_{j} * {U}_{j} ( {ς}_{i} , : ) - {U}_{j} ( i , : ) ‖}_{p} \} . \qquad ( 3 )$$

Equation (3) denotes a constraint condition of the spatial filter Wⱼ, ∥*∥ₚ is the p-norm of a vector, the argmin function is to find a variable value that minimizes the target function, and Ŵⱼ is an estimation of the spatial filter Wⱼ under this constraint condition.

In the equation, Uⱼ(i,:) is the template signal, representing a signal of the target channel i corresponding to the time slice Uⱼ; Uⱼ(ç₁,:) is the fitting signal, representing a signal of the channel set çᵢ corresponding to the time slice Uᵢ, çᵢ is a channel set having any number of channels that are arbitrarily selected from (N_{c}−1) remaining channels except the target channel i.

4) The spatial filter is applied to spatially filter the signals corresponding to two time slices to obtain augmented signals of different time slices of the piece of segmented data respectively. The application process of the spatial filter can be denoted as follows:

χⱼ=Ŵⱼ*Uⱼ(çᵢ,:)−Uⱼ(i,:), (4)

γⱼ=Ŵⱼ*Vⱼ(çᵢ,:)−Vⱼ(i,:) (5)

In equations (4) and (5), χⱼ∈R^{1×m} and γⱼ∈R^{1×n} represent the augmented signals obtained for the Uⱼ and Vⱼ respectively, and the number of all possible values of these two augmented signals are consistent with the number of combination manners corresponding to the selection channel set çᵢ, Vⱼ(i,:) denotes the signal of the target channel i corresponding to the time slice Vⱼ, and Vⱼ (çᵢ,:) denotes the signal of the channel set çᵢ corresponding to the time slice Vⱼ.

5) The above 3) and 4) are repeated until all pieces of segmented data are processed. All the segmented augmented signals obtained are spliced and integrated, and a final augmented signal of the target channel and channel set is output.

Total j pieces of segmented data within different time windows are traversed to obtain segmented augmented signals χⱼ∈R^{1×m} and γⱼ∈R^{1×n} of the segmented data. The segmented augmented signals of different time windows are spliced and integrated based on the set of center points {t⁽¹⁾, t⁽²⁾ . . . t^{(j)}} of the time window, and a new component signal y∈R^{1×N}^{ₜ} (i.e., the augmented signal) of the target channel i and the channel set çᵢ is obtained.

When tₛ<Δt₁+Δt₂, superimposition and average processing can be performed on an overlapping part. When tₛ≥Δt₁+Δt₂, zero-padding or interpolation processing can be performed on a missing part. It is to be noted that when the signal corresponding to the time slice \textcircled{1} within [t−Δt₁,t] is a task-independent signal, while the signal corresponding to the time slice \textcircled{2} within [t,t+Δt₂] is a task-related signal, only parts of the signal meeting γⱼ∈R^{1×n} can be used for splicing and integrating.

The above 2) to 5) are repeated to traverse all combination manners between the target channel and the selection channel set to obtain several new component signals. These new component signals together with the original signal constitute a new EEG component space.

In detail, each channel can be used as the target channel i in turn and each corresponding channel set can be used as the channel set çᵢ in turn to repeat the above 2) to 5) to obtain several new component signals y⁽ⁿ⁾∈R^{1×N}^{ₜ}, where y⁽ⁿ⁾ represents a n^{th} new component signal. The set {y⁽¹⁾, y⁽²⁾ . . . y⁽ⁿ⁾} or its subset together with the original signal can form a new EEG component space Y∈R^{N}^{ₛ}^{×N}^{ₜ}, where Nₛ denotes the number of components in an augmented space, and its maximum possible value can be determined by a following equation (6):

Nₛ=N_{c}×(2^{N}^{_{c}}⁻¹+1). (6)

The disclosure has a wide range of applications in EEG signal processing and analysis, and has considerable practicability. The obtained augmentation signals are used as new EEG components, to map the original data into a new component space, thereby effectively exploring potential EEG information.

In addition, the augmented signals are obtained by constructing and applying the dynamic spatial filter, such that the obtained augmented signals can reflect the current EEG characteristics, thereby improving reliability and validity.

The method for augmenting an EEG signal according to embodiments of the disclosure can generate a large number of augmented signals, such that the obtained new component space includes the original signal and the augmented signals. The above can be described in detail as follows. For one target channel, totally 2^{N}^{_{c}}⁻¹ channel sets çᵢ can be determined, where each channel set includes any number of channels that are arbitrarily selected from N_{c}−1 remaining channels. For signals of N_{c} channels, the method can generate N_{c}×2^{N}^{_{c}}⁻¹ augmented signals. The generated augmented signals or its subset together with the original signal can form the new EEG component space.

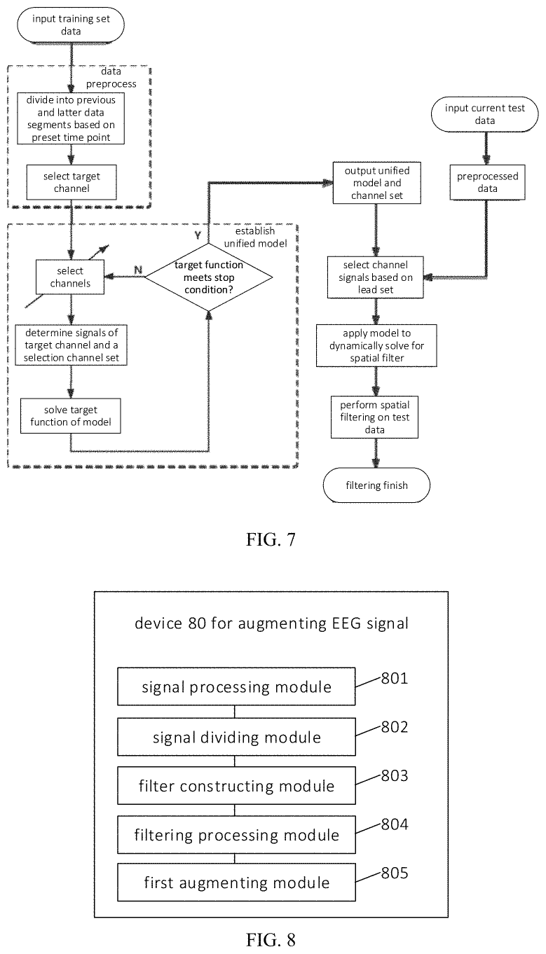

FIG. 7 is a schematic diagram according to another embodiment of the disclosure. As illustrated in FIG. 7, a method for dynamically constructing an EEG spatial filter can include the following.

At block 101, training set data is divided into a previous data segment and a latter data segment based on a preset time point after inputting the training set data. A target channel is selected.

That is, the block 101 is to realize the preprocessing of the training set data.

At block 102, signals of the target channel and the selection channel set corresponding to the two time slices are determined by selecting a part of channels from remaining channels. A target function is solved through the above-mentioned four signals to construct a unified model. For example, the target channel is Oz, and three channels POz, Pz, and FCz are selected in an initial stage to form the channel set {POz, Pz, FCz}, where elements of the channel set are names of the respective channels. Within the previous and latter time slices, signals of the target channel Oz and the channel set {POz, Pz, FCz} are extracted as the four signals.

At block 103, it is determined whether an output of the target function meets a stop condition. In response to meeting the stop condition, a block 104 is executed. In response to not meeting the stop condition, the block 102 is executed again.

That is, the establishment of the unified model is achieved through the above blocks 102 and 103. When the stop condition is not met, the channel set is re-selected again.

At block 104, current test data is input and pre-processed according to the block 101, signals can be extracted from the pre-processed test data based on the channel set obtained through the unified model. For example, in a case where the target channel is Oz, and the channel set obtained by the unified model is {POz, Pz, FCz}, signals of the three channels POz, Pz, FCz are also extracted from the test data as the signal of the channel set.

At block 105, a model is applied to the signal of the target channel in combination with the signals selected in block 104, to dynamically solve for the spatial filter suitable for the current environment, and perform spatial filtering on the test data, until the filtering is completed.

In conclusion, through the above blocks 101 to 105, the dynamic construction of the EEG spatial filter is realized, which meets various needs in practical applications.

The technical solution will be further expanded and refined in conjunction with specific calculation equations, examples, and FIG. 7 as follows.

All trial signals in the training data under a certain stimulus condition can be expressed as a three-dimensional tensor ϕ∈R^{N}^{_{c}}^{×N}^{ₛ}^{×N}^{ₜ}, N_{c} denotes the number of channels included in the collected data, Nₛ denotes the total number of trials and Nₜ denotes the number of sampling points of this data segment. According to a preset starting time (t=t₀), the tensor ϕ is divided into an EEG segment X∈R^{N}^{_{c}}^{×N}^{ₛ}^{×m} within t<t₀ and an EEG segment Y∈R^{N}^{_{c}}^{×N}^{ₛ}^{×n} within t>t₀, where m and n are the numbers of data points and are constants respectively, R denotes a set of constants.

A relationship between the two EEG segments X and Y is used to model and design a spatial filter, to perform filtering and noise reduction processing on the EEG segment within t>t₀. The above includes the following.

(1) A unified model G is established from the training data to solve for a dynamic filter, see equations (1)-(4).

$$\{ \begin{matrix}{\hat{U}}_{i}^{( k )} = {argmin}_{{U}_{i}^{( k )}} \{ {‖ {U}_{i}^{( k )} * {X}^{( k )} ( {ς}_{i} , : ) - {X}^{( k )} ( i , : ) ‖}_{p} \} & ( 1 ) \\ {\hat{ς}}_{i} = {argmax}_{{ς}_{i}} \{ f ( \{ {𝒳}^{( k )} \vert k = 1 , 2 , … {N}_{s} \} , \{ {γ}^{( k )} \vert k = 1 , 2 , … {N}_{s} \} ) \} & ( 2 ) \\ {𝒳}^{( k )} = {U}_{i}^{( k )} * {X}^{( k )} ( {ς}_{i} , : ) - {X}^{( k )} ( i , : ) & ( 3 ) \\ {γ}^{( k )} = {U}_{i}^{( k )} * {Y}^{( k )} ( {ς}_{i} , : ) - {Y}^{( k )} ( i , : ) & ( 4 )\end{matrix}$$

where, X^{(k)}(i,:)∈R^{1×m} and Y^{(k)}(i,:)∈R^{1×n} denote signals of the target channel i of a k^{th} trial within the two time slices t<t₀ and t>t₀ respectively; çᵢ denotes a channel set including φ (the value of φ is not fixed) channels selected from the remaining channels except the target channel i, X^{(k)}(çᵢ,:)∈R^{φ×m} and Y^{(k)}(çᵢ,:)∈R^{φ×n} denote signals of the channel set çᵢ of the k^{th} trial within the two time slices before and after t₀ respectively.

The equation (1) is a constraint condition of the spatial filter Uᵢ^{(k)}, ∥*∥_{P} denotes p-norm of a vector, the argmin function is to search for a variable value that minimizes the value of the target function, the argmax function is to search for a variable value that maximizes the value of the target function. The equation (2) is a target function ƒ used for determining the channel set çᵢ, and its output is a quantitative index related to the signal quality, with various specific forms, including but not limited to the spectrum, energy, signal-to-noise ratio of the characteristic signal, and so on. Inputs of the target function χ^{(k)}∈R^{1×m} and γ^{(k)}∈R^{1×n} are obtained by solving the equations (3) and (4).

(2) The test data is preprocessed by dividing the test data into data segments X̅∈R^{N}^{_{c}}^{×N}^{ₛ}^{×m} and Y̅∈R^{N}^{_{c}}^{×N}^{ₛ}^{×n} within time slices before and after a time point (t=t₀). Signals X̅(çᵢ,:)∈R^{φ×m}, X̅(i,:)∈R^{1×m}, Y̅(çᵢ,:)∈R^{φ×n} and Y̅(i,:)∈R^{1×n} are obtained based on the channel set çᵢ and the target channel i that are obtained through the unified model G, where X̅(i,:)∈R^{1×m} and X̅(çᵢ,:)∈R^{φ×m} respectively denote the single-trial signal of the target channel i and the single-trial signal of the channel set çᵢ in the test data within the time slice t<t₀ respectively; Y̅(i,:)∈R^{1×n} and Y̅(çᵢ,:)∈R^{φ×n} denote the single-trial signal of the target channel i and the single-trial signal of the channel set çᵢ in the test data within the time slice t>t₀, respectively.

The spatial filter Wᵢ suitable for the current EEG environment is dynamically solved based on the equation (1) of the unified model G, and the details can be seen in a following equation (5):

$${\hat{W}}_{i} = {argmin}_{{W}_{i}} \{ {‖ {W}_{i} * \overline{X} ( {ς}_{i} , : ) - \overline{X} ( i , : ) ‖}_{p} \} . \qquad ( 5 )$$

(3) The spatial filtering processing is performed on the current test data using the equation (4) of the unified model G and the spatial filter Wᵢ to obtain a noise-reduced signal γ̅, see a following equation (6), where γ̅ denotes the test signal within the time slice t>t₀ after the filtering is completed:

γ̅=Wᵢ*Y̅(çᵢ,:)−Y̅(i,:). (6)

In theory, any one of the channels included in the training data can be used as the target channel to perform the above blocks and establish the unified model, to realize the spatial filtering on all channels of the test data.

FIG. 8 is a schematic flowchart according to another embodiment of the disclosure.

As illustrated in FIG. 8, the augmented device 80 of an EEG signal includes: a signal obtaining module 801, a signal dividing module 802, a filter constructing module 803, a filtering processing module 804, and a first augmenting module 805. The signal obtaining module 801 is configured to obtain an EEG signal, determine a first channel from a plurality of channels included in the EEG signal, determine a second channel set including at least one second channel selected from remaining channels except the first channel, and gather the first channel and the second channel set as a current combination manner.

The signal dividing module 802 is configured to divide the EEG signal into a plurality of segmented EEG signals, and divide a segmented EEG signal into a signal corresponding to a first time slice and a signal corresponding to a second time slice.

The filter constructing module 803 is configured to determine the signals of the first channel corresponding to the first time slice as first signals, determine the signals of the second channel set corresponding to the first time slice as second signals, and construct spatial filters based on the first signals and the second signals respectively.

The filtering processing module 804 is configured to perform spatial filtering processing on the signals corresponding to the first time slice and the signals corresponding to the second time slice by respective spatial filters to obtain augmented signals.

The first augmenting module 805 is configured to splice and integrate the augmented signals of the plurality of segmented EEG signals to augment the EEG signal.

In some embodiments, the device 80 further includes a second augmenting module. The second augmenting module is configured to update the current combination manner and augment the EEG signal corresponding to the updated current combination manner, after augmenting the EEG signal by splicing and integrating a plurality of augmented signals corresponding to the plurality of segmented EEG signals.

In some embodiments, the signal dividing module 802 includes a signal dividing submodule. The signal dividing submodule is configured to divide the EEG signal into the plurality of segmented EEG signals through a dynamic time window. The dynamic time window is denoted by a time range [t−Δt₁,t+Δt₂] centered on t, where [t−Δt₁,t] denotes a first time slice, and [t,t+Δt₂] denotes a second time slice.

In some embodiments, the filtering constructing module 803 is configured to construct the spatial filter with a target equation, where the spatial filter is represented by:

$${\hat{W}}_{j} = {argmin}_{{W}_{j}} \{ {‖ {W}_{j} * {U}_{j} ( {ς}_{i} , : ) - {U}_{j} ( i , : ) ‖}_{p} \} ,$$

where, the target equation denotes a constraint condition of the spatial filter Wⱼ corresponding to the segmented EEG signal having a serial number of j, ∥*∥ₚ denotes a p-norm of a vector, the argmin function is to search for a variable value that minimizes the target function, Ŵⱼ is an estimation of the spatial filter Wⱼ under the constraint condition, Uⱼ(i,:) is the first signal, i denotes the serial number of the first channel; Uⱼ(çᵢ,:) is the second signal, çᵢ denotes the second channel set, Uⱼ∈R^{N}^{_{c}}^{×m} denotes the signal corresponding to the first time slice of the segmented EEG signal having the serial number of j, N_{c} denotes the number of channels included in the EEG signal, m denotes the number of sampling points within the dynamic time window, m=[Δt₁×Fₛ], m is an integer not exceeding the real number, and Fₛ is a sampling frequency of the EEG signal.

In some embodiments, the filtering processing module 804 is configured to obtain the augmented signal through following equations:

χⱼ=Ŵⱼ*Uⱼ(çᵢ,:)−Uⱼ(i,:), and

γⱼ=Ŵⱼ*Vⱼ(çᵢ,:)−Vⱼ(i,:),

where, χⱼ∈R^{1×m} and γⱼ∈R^{1×n} denotes the augmented signals obtained by filtering Uⱼ and Vⱼ respectively, Vⱼ∈R^{N}^{_{c}}^{×n} denotes the signal corresponding to the second time slice of the segmented EEG signal having the serial number of j, n represents the number of sampling points within the dynamic time window, n=[Δt₂×Fₛ], n denotes an integer not exceeding the real number, Vⱼ(i,:) denotes the signal of the first channel having the serial number of i corresponding to the second time slice of the segmented EEG signal having the serial number of j, and Vⱼ (çᵢ,:) denotes the signal of the second channel set corresponding to the second time slice of the segmented EEG signal having the serial number of j, where the second channel set corresponds to the first channel having the serial number of i.

It is to be noted that, the foregoing explanations of the method for augmenting an EEG signal is also applicable to the device of this embodiment, which will not be repeated here.

According to embodiments of the disclosure, there is further provided an electronic device and a readable storage medium.

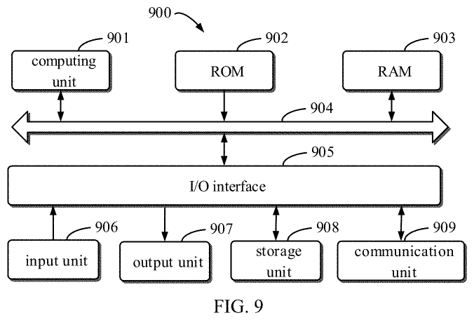

FIG. 9 is a block diagram illustrating an electronic device that can be used to implement the method for augmenting an EEG signal according to embodiments of the disclosure. The electronic device is intended to represent various forms of digital computers, such as laptop computers, desktop computers, workstations, personal digital assistants, servers, blade servers, mainframe computers, and other suitable computers. The electronic device may also represent various forms of mobile devices, such as personal digital processors, cellular phones, smart phones, wearable devices, and other similar computing devices. The components shown herein, their connections and relationships, and their functions are by way of example only, are not intended to limit implementations of the disclosure described and/or claimed herein.

As illustrated in FIG. 9, the device 900 includes a computing unit 901 that can be configured to execute various appropriate actions and operations according to a computer program stored in a read only memory (ROM) 902 or loaded into a random access memory (RAM) 903 from a storage unit 908. In the RAM 903, various programs and data necessary for the operation of the device 900 can also be stored. The computing unit 901, the ROM 902, and the RAM 903 are connected to each other through a bus 904. An input/output (I/O) interface 905 is also connected to bus 904.

Various components in the device 900 are connected to the I/O interface 905, including: an input unit 906, such as a keyboard, mouse, etc.; an output unit 907, such as various types of displays, speakers, etc.; a storage unit 908, such as a magnetic disk, an optical disk, etc.; and a communication unit 909, such as a network card, a modem, a wireless communication transceiver, and the like. The communication unit 909 allows the device 900 to exchange information/data with other devices through a computer network such as the Internet and/or various telecommunication networks.

Computing unit 901 may be various general-purpose and/or special-purpose processing components with processing and computing capabilities. Some examples of computing units 901 include, but are not limited to, central processing units (CPUs), graphics processing units (GPUs), various specialized artificial intelligence (AI) computing chips, various computing units that run machine learning model algorithms, digital signal processing processor (DSP), and any suitable processor, controller, microcontroller, etc. The computing unit 901 performs the various methods and processes described above, for example, the method for augmenting an EEG signal.

For example, in some embodiments, the method for augmenting an EEG signals may be implemented as a computer software program tangibly embodied on a machine-readable medium, such as storage unit 908. In some embodiments, part or all of the computer program may be loaded and/or installed on device 900 via ROM 902 and/or communication unit 909. When the computer program is loaded into the RAM 903 and executed by the computing unit 901, one or more steps of the above-described method for augmenting an EEG signal may be performed. Alternatively, in other embodiments, the computing unit 901 may be configured by any other suitable means (e.g., by means of firmware) to perform the method for augmenting the EEG signal.

Various implementations of the systems and techniques described herein above may be implemented in digital electronic circuitry, integrated circuit systems, field programmable gate arrays (FPGAs), application specific integrated circuits (ASICs), application specific standard products (ASSPs), systems on chips system (SOC), load programmable logic device (CPLD), computer hardware, firmware, software, and/or combinations thereof. These various embodiments may include being implemented in one or more computer programs executable and/or interpretable on a programmable system including at least one programmable processor that may be a special purpose or general-purpose programmable processor, may receive data and instructions from a storage system, at least one input device, and at least one output device, and transmit data and instructions to the storage system, the at least one input device, and the at least one output device.

The program code for implementing the method for augmenting an EEG signal of the disclosure may be written in any combination of one or more programming languages. These program codes may be provided to a processor or controller of a general purpose computer, special purpose computer, or other programmable device for augmenting an EEG signal, such that the program code, when executed by the processor or controller, causes the flowcharts and/or block diagrams to execute the specified functions/operations. The program code may execute entirely on the machine, partly on the machine, partly on the machine and partly on a remote machine as a stand-alone software package or entirely on the remote machine or server.

In the context of this disclosure, a machine-readable medium may be a tangible medium that may contain or store the program for use by or in connection with the instruction execution system, apparatus or device. The machine-readable medium may be a machine-readable signal medium or a machine-readable storage medium. Machine-readable media may include, but are not limited to, electronic, magnetic, optical, electromagnetic, infrared, or semiconductor systems, devices, or devices, or any suitable combination of the foregoing. More specific examples of machine-readable storage media would include one or more wire-based electrical connections, portable computer disks, hard disks, random access memory (RAM), read only memory (ROM), erasable programmable read only memory (EPROM or flash memory), fiber optics, compact disk read only memory (CD-ROM), optical storage devices, magnetic storage devices, or any suitable combination of the foregoing.

To provide interaction with a user, the systems and techniques described herein may be implemented on a computer having a display device (e.g., a CRT (cathode ray tube) or LCD (liquid crystal display) monitor) for displaying information to the user); and a keyboard and pointing device (e.g., a mouse or trackball) through which a user can provide input to the computer. Other kinds of devices can also be used to provide interaction with the user; for example, the feedback provided to the user can be any form of sensory feedback (e.g., visual feedback, auditory feedback, or tactile feedback); and can be in any form (including acoustic input, voice input, or tactile input) to receive input from the user.

The systems and techniques described herein can be implemented on a computing system that includes back-end components (e.g., as a data server), or a computing system that includes middleware components (e.g., an application server), or a computing system that includes front-end components (e.g., a user computer having a graphical user interface or web browser through which a user can interact with implementations of the systems and techniques described herein), or including such backend components, middleware components, or any combination of front-end components in a computing system. The components of the system may be interconnected by any form or medium of digital data communication (e.g., a communication network). Examples of communication networks include Local Area Networks (LANs), Wide Area Networks (WANs), the Internet, and blockchain networks.

A computer system can include clients and servers. Clients and servers are generally remote from each other and usually interact through a communication network. The relationship of client and server arises by computer programs running on the respective computers and having a client-server relationship to each other. The server can be a cloud server, also known as a cloud computing server or a cloud host. It is a host product in the cloud computing service system to solve the problem of traditional physical hosts and VPS services (Virtual Private Server). There are the defects of difficult management and weak business expansion. The server can also be a server of a distributed system, or a server combined with a blockchain.

It should be noted that, in the description of the disclosure, the terms “first”, “second”, etc. are only used for the purpose of description, and should not be construed as indicating or implying relative importance. Also, in the description of this application, unless otherwise specified, “plurality” means two or more.

Any description of a process or method in the flowcharts or otherwise described herein may be understood to represent a module, segment or portion of code comprising one or more executable instructions for implementing a specified logical function or step of the process, and the scope of the preferred embodiments of the disclosure includes alternative implementations in which the functions may be performed out of the order shown or discussed, including performing the functions substantially concurrently or in the reverse order depending upon the functions involved, which should be understood by those skilled in the art to which the embodiments of the disclosure belong.

It should be understood that various parts of this disclosure may be implemented in hardware, software, firmware, or a combination thereof. In the above-described embodiments, various steps or methods may be implemented in software or firmware stored in memory and executed by a suitable instruction execution system. For example, if implemented in hardware, as in another embodiment, it can be implemented by any one or a combination of the following techniques known in the art, such as Discrete logic circuits, ASICs with suitable combinational logic gates, Programmable Gate Arrays (PGA), Field Programmable Gate Arrays (FPGA), etc.

Those skilled in the art can understand that all or part of the steps carried by the methods of the above embodiments can be completed by instructing the relevant hardware through a program, and the program can be stored in a computer-readable storage medium, and the program can be stored in a computer-readable storage medium. When executed, one or a combination of the steps of the method embodiment is included.

In addition, each functional unit in each embodiment of the disclosure may be integrated into one processing module, or each unit may exist physically alone, or two or more units may be integrated into one module. The above-mentioned integrated modules can be implemented in the form of hardware, and can also be implemented in the form of software function modules. If the integrated modules are implemented in the form of software functional modules and sold or used as independent products, they may also be stored in a computer-readable storage medium.

The above-mentioned storage medium may be a read-only memory, a magnetic disk or an optical disk, and the like.

In the description of this specification, description with reference to the terms “one embodiment,” “some embodiments,” “example,” “specific example,” or “some examples”, etc., mean specific features described in connection with the embodiment or example, structure, material or feature is included in at least one embodiment or example of the disclosure. In this specification, schematic representations of the above terms do not necessarily refer to the same embodiment or example. Furthermore, the particular features, structures, materials or characteristics described may be combined in any suitable manner in any one or more embodiments or examples.

Although the embodiments of the disclosure have been shown and described above, it should be understood that the above embodiments are exemplary and should not be construed as limitations to the disclosure. Embodiments are subject to variations, modifications, substitutions and variations.

1. A method for performing spatial filtering and augmenting an electroencephalogram (EEG) signal, including:
constructing, by a processor, a spatial filter based on channel information of the EEG signal; and
augmenting, by the processor, the EEG signal with the spatial filter.

2. The method of claim 1, wherein constructing the spatial filter based on the channel information of the EEG signal comprises:
dividing training set data into a previous data segment corresponding to a first time slice and a latter data segment corresponding to a second time slice based on a preset time point after inputting the training set data, and selecting a target channel from the previous data segment and the latter data segment;
obtaining a selection channel set by selecting a part of channels from remaining channels, determining four signals of the target channel and the selection channel set corresponding to the first time slice and the second time slice respectively, and constructing a unified model based on a target function and the four signals;
in response to determining an output of the target function meets a stop condition, preprocessing current test data by dividing the current test data into a previous data segment and a latter data segment based on a preset time point to obtain pre-processed test data after inputting the current test data, and selecting signals from the pre-processed test data based on the selection channel set output by the unified model;
applying a model on a test signal of the target channel in combination with the signals selected from the pre-processed test data, dynamically obtaining the spatial filter suitable for a current environment, and performing spatial filtering on the current test data; and
in response to determining that the output of the target function does not meet the stop condition, re-selecting a part of channels, re-determining four signals and re-constructing the unified model.

3. The method of claim 2, wherein dividing the training set data into the previous data segment corresponding to a first time slice and the latter data segment corresponding to a second time slice based on the preset time point comprises:
dividing a tensor ϕ based on a preset start time t=t₀ into an EEG segment X∈R^{N}^{_{c}}^{×N}^{ₛ}^{×m} within the first time slice t<t₀ and an EEG signal Y∈R^{N}^{_{c}}^{×N}^{ₛ}^{×n} within the second time slice t>t₀, where m and n are number of data points and are constants, R denotes a set of constants, N_{c} denotes the number of channels contained in collected data, Nₛ denotes a total number of trials.

4. The method of claim 3, wherein the unified model is expressed by:
$$\{ \begin{matrix}{\hat{U}}_{i}^{( k )} = {argmin}_{{U}_{i}^{( k )}} \{ {‖ {U}_{i}^{( k )} * {X}^{( k )} ( {ς}_{i} , : ) - {X}^{( k )} ( i , : ) ‖}_{p} \} & ( 1 ) \\ {\hat{ς}}_{i} = {argmax}_{{ς}_{i}} \{ f ( \{ {𝒳}^{( k )} \vert k = 1 , 2 , … {N}_{s} \} , \{ {γ}^{( k )} \vert k = 1 , 2 , … {N}_{s} \} ) \} & ( 2 ) \\ {𝒳}^{( k )} = {U}_{i}^{( k )} * {X}^{( k )} ( {ς}_{i} , : ) - {X}^{( k )} ( i , : ) & ( 3 ) \\ {γ}^{( k )} = {U}_{i}^{( k )} * {Y}^{( k )} ( {ς}_{i} , : ) - {Y}^{( k )} ( i , : ) & ( 4 )\end{matrix}$$
where, X^{(k)}(i,:)∈R^{1×m} and Y^{(k)}(i,:)∈R^{1×n} denote signals of the target channel i of a k^{th} trial within the two time slices t<t₀ and t>t₀ respectively; çᵢ denotes the channel set including φ channels selected from the remaining channels except the target channel i, X^{(k)}(çᵢ,:)∈R^{φ×m} and Y^{(k)}(çᵢ,:)∈R^{φ×n} denote signals of the channel set çᵢ of a k^{th} trial within the two time slices before and after t₀ respectively, \hat{ç}ᵢ denotes an estimation of the channel set çᵢ that maximizes an output value of a function ƒ;
an equation (1) is a constraint condition of the spatial filter Uᵢ^{(k)}, ∥*∥ₚ denotes p-norm of a vector, argmin denotes searching for a variable value that minimizes a value of the target function, argmax denotes searching for a variable value that maximizes the value of the target function; Ûᵢ^{(k)} denotes an estimation of the spatial filter Uᵢ^{(k)} that minimizes an output value of a corresponding p-norm;
an equation (2) is the target function ƒ for determining the channel set çᵢ, and an output of the target function is a quantitative index related to a signal quality, and inputs of the target function χ^{(k)}∈R^{1×m} and γ^{(k)}∈R^{1×n} are obtained by solving the equations (3) and (4).

5. The method of claim 4, wherein applying the model on the test signal of the target channel, dynamically obtaining the spatial filter suitable for the current environment, and performing the spatial filtering on the current test data comprises:
$$\hat{{W}_{i}} = {argmin}_{{W}_{i}} \{ {‖ {W}_{i} * \overline{X} ( {ς}_{i} , : ) - \overline{X} ( i , : ) ‖}_{p} \} , \qquad ( 5 )$$
where the equation (4) and the spatial filter Wᵢ are used to perform the spatial filtering on current test data to obtain a noise-reduced signal γ̅ which is denoted by an equation (6), where γ̅ denotes the test signal within the second time slice t>t₀ after the filtering is completed:
γ̅=Wᵢ*Y̅(çᵢ,:)−Y̅(i,:), (6)
where, Ŵᵢ is an estimation of the spatial filter that minimizes an output value of the p-norm, X̅(çᵢ,:) and X̅(i,:) denote a single-trial signal of the channel set çᵢ in the test data within the first time slice t<t₀ and a single-trial signal of the target channel i in the test data within the first time slice t<t₀ respectively, Y̅(çᵢ,:) and Y̅(i,:) denote a single-trial signal of the channel set çᵢ in the test data within the second time slice t>t₀ and a single-trial signal of the target channel i in the test data within the second time slice t>t₀ respectively.

6. The method of claim 1, wherein constructing the spatial filter based on the channel information of the EEG signal comprises:
obtaining the EEG signal, determining a first channel from a plurality of channels contained in the EEG signal, determining a second channel set containing at least one channel selecting from remaining channels except the first channel; and gathering the first channel and the second channel set as a current combination manner;
dividing the EEG signal into a plurality of segmented EEG signals, and dividing each of the plurality of segmented EEG signals into a signal corresponding to a first time slice and a signal corresponding to a second time slice; and
determining signals of the first channel corresponding to the first time slice as first signals, determining signals of the second channel set corresponding to the first time slice as second signals, and constructing a plurality of spatial filters based on the first signals and the second signals respectively.

7. The method of claim 6, wherein augmenting the EEG signal by the spatial filter comprises:
performing spatial filtering processing on the signals corresponding to the first time slice and the signals corresponding to the second time slice with the plurality of spatial filters to obtain augmented signals; and
splicing and integrating the augmented signals corresponding to the plurality of segmented EEG signals to augment the EEG signal.

8. The method of claim 7, after splicing and integrating the plurality of augmented signals corresponding to the plurality of segmented EEG signals to augment the EEG signal, further comprising:
updating the current combination manner, and augmenting the EEG signal corresponding to an updated combination manner.

9. The method of claim 7, wherein dividing the EEG signal into the plurality of segmented EEG signals, and dividing each of the plurality of segmented EEG signals into the signal corresponding to the first time slice and the signal corresponding to the second time slice comprises:
dividing the EEG signal into the plurality of segmented EEG signals by a dynamic time window, where the dynamic time window is a time range [t−Δt₁,t+Δt₂] centered on t, [t−Δt₁,t] denotes the first time slice, and [t,t+Δt₂] denotes the second time slice.

10. The method of claim 9, wherein the spatial filter is constructed through a target equation, and the target equation is expressed by:
$${\hat{W}}_{j} = {argmin}_{{W}_{j}} \{ {‖ {W}_{j} * {U}_{j} ( {ς}_{i} , : ) - {U}_{j} ( i , : ) ‖}_{p} \}$$
where, the target equation denotes a constraint condition of the spatial filter Wⱼ corresponding to the segmented EEG signal having a serial number of ∥*∥ₚ denotes p-norm of a vector, argmin function is to search for a variable value that minimizes a target function, Ŵ₁ denotes an estimation of the spatial filter Wⱼ under the constraint condition, Uⱼ(i,:) denotes the first signal, i denotes a serial number of the first channel, Uⱼ(çi,:) denotes the second signal, çᵢ denotes the serial number of the second channel set, Uⱼ∈R^{N}^{_{c}}^{×m} denotes the signal corresponding to the first time slice of the segmented EEG signal having the serial number of j, N_{c} denotes the number of channels contained in the segmented EEG signal, m denotes the number of sampling points within the dynamic time window, m=[Δt₁×Fₛ], m is an integer not exceeding a real number, Fₛ denotes a sampling frequency of the EEG signal.

11. The method of claim 10, wherein performing the spatial filtering processing on the signals corresponding to the first time slice and the signals corresponding to the second time slice of the segmented EEG signals with the spatial filter to obtain augmented signals comprising:
obtaining the augmented signals by:
χⱼ=Ŵⱼ*Uⱼ(çᵢ,:)−Uⱼ(i,:); and
γⱼ=Ŵⱼ*Vⱼ(çᵢ,:)−Vⱼ(i,:),
where, χ₁∈R^{1×m} and γⱼ∈R^{1×n} denote the augmented signals obtained by performing filter processing on Uⱼ and Vⱼ respectively, Vⱼ∈R^{N}^{_{c}}^{×n} denotes the signals corresponding to the second time slice of the segmented EEG signal having a serial number of j, n denotes the number of sampling data within the dynamic time window, n=[Δt₂×Fₛ], n is an integer not exceeding a real number, Vⱼ(i,:) denotes the signal of the first channel having the serial number of i corresponding to the second time slice of the segmented EEG signal having the serial number of j, and Vⱼ(çᵢ,:) denotes the signal of the second channel set corresponding to the second time slice of the segmented EEG signal having the serial number of j, the second channel set corresponds to the target channel having the serial of i.

12.-14. (canceled)

15. An electronic device, comprising:
at least one processor; and
a memory, communicating with the at least one processor,
wherein the memory is configured to store instructions executable by the at least one processor, and when the instructions are executed by the at least one processor, the at least one processor is configured to:
construct a spatial filter based on channel information of an electroencephalogram (EEG) signal; and
augment the EEG signal with the spatial filter.

16. A non-transitory computer-readable storage medium having computer instructions stored thereon, wherein the computer instructions are configured to cause a computer to execute a method for performing spatial filtering and augmenting an electroencephalogram (EEG) signal, the method comprising:
constructing a spatial filter based on channel information of the EEG signal; and
augmenting the EEG signal with the spatial filter.

17. The electronic device of claim 15, wherein the at least one processor is configured to:
obtain the EEG signal, determine a first channel from a plurality of channels contained in the EEG signal, determine a second channel set containing at least one channel selecting from remaining channels except the first channel; and gather the first channel and the second channel set as a current combination manner;
divide the EEG signal into a plurality of segmented EEG signals, and divide each of the plurality of segmented EEG signals into a signal corresponding to a first time slice and a signal corresponding to a second time slice; and
determine signals of the first channel corresponding to the first time slice as first signals, determine signals of the second channel set corresponding to the first time slice as second signals, and construct a plurality of spatial filters based on the first signals and the second signals respectively.

18. The electronic device of claim 17, wherein the at least one processor is configured to:
perform spatial filtering processing on the signals corresponding to the first time slice and the signals corresponding to the second time slice with the plurality of spatial filters to obtain augmented signals; and
splice and integrate the augmented signals corresponding to the plurality of segmented EEG signals to augment the EEG signal.

19. The electronic device of claim 18, wherein the at least one processor is further configured to:
update the current combination manner, and augment the EEG signal corresponding to an updated combination manner.

20. The electronic device of claim 18, wherein the at least one processor is configured to:
divide the EEG signal into the plurality of segmented EEG signals by a dynamic time window, where the dynamic time window is a time range [t−Δt₁,t+Δt₂] centered on t, [t−Δt₁,t] denotes the first time slice, and [t,t+Δt₂] denotes the second time slice.

21. The electronic device of claim 20, wherein the at least one processor is configured to construct the spatial filter through a target equation, and the target equation is expressed by:
$${\hat{W}}_{j} = {argmin}_{{W}_{j}} \{ {‖ {W}_{j} * {U}_{j} ( {ς}_{i} , : ) - {U}_{j} ( i , : ) ‖}_{p} \}$$
where, the target equation denotes a constraint condition of the spatial filter Wⱼ corresponding to the segmented EEG signal having a serial number of j, ∥*∥ₚ denotes p-norm of a vector, argmin function is to search for a variable value that minimizes a target function, Ŵⱼ denotes an estimation of the spatial filter Wⱼ under the constraint condition, Uⱼ(i,:) denotes the first signal, i denotes a serial number of the first channel, Uⱼ(çᵢ,:) denotes the second signal, çᵢ denotes the serial number of the second channel set, Uⱼ∈R^{N}^{_{c}}^{×m} denotes the signal corresponding to the first time slice of the segmented EEG signal having the serial number of j, N_{c} denotes the number of channels contained in the segmented EEG signal, m denotes the number of sampling points within the dynamic time window, m=[Δt₁×Fₛ], m is an integer not exceeding a real number, Fₛ denotes a sampling frequency of the EEG signal.

22. The electronic device of claim 21, wherein the at least one processor is configured to:
obtain the augmented signals by:
χⱼ=Ŵⱼ*Uⱼ(çᵢ,:)−Uⱼ(i,:); and
γⱼ=Ŵⱼ*Vⱼ(çᵢ,:)−Vⱼ(i,:),
where, χⱼ∈R^{1×m} and γⱼ∈R^{1×n} denote the augmented signals obtained by performing filter processing on Uⱼ and Vⱼ respectively, Vⱼ∈R^{N}^{_{c}}^{×n} denotes the signals corresponding to the second time slice of the segmented EEG signal having a serial number of j, n denotes the number of sampling data within the dynamic time window, n=[Δt₂×Fₛ], n is an integer not exceeding a real number, Vⱼ(i,:) denotes the signal of the first channel having the serial number of i corresponding to the second time slice of the segmented EEG signal having the serial number of j, and Vⱼ(çᵢ,:) denotes the signal of the second channel set corresponding to the second time slice of the segmented EEG signal having the serial number of j, the second channel set corresponds to the target channel having the serial of i.
